(19) United States
(12) Patent Application Publication
(10) Pub. No.: US 2024/0197007 A1
(43) Pub. Date: Jun. 20, 2024

(54) PORTABLE AIR SANITATING DEVICE

(71) Applicant: James Joseph WADE, Liberty Lake, WA (US)

(72) Inventor: James Joseph WADE, Liberty Lake, WA (US)

(21) Appl. No.: 18/084,094

(22) Filed: Dec. 19, 2022

(51) Int. Cl. A41D 13/11; A61L 9/20; A62B 18/00

(52) U.S. Cl. CPC A41D 13/1184; A61L 9/20; A62B 18/003; A61L 2209/15

(57) ABSTRACT

This invention is directed to a portable air sanitizing device that sterilizes airborne pathogen in the ambient air using UVC light and provides the sanitized air as a cone or cylinder of high velocity air to prevent mixing with untreated ambient air. In one embodiment, a nozzle directs the cone or cylinder of high velocity sanitized air at a user's mouth and nose so that the user can breathe in the sanitized air before it mixes with the untreated ambient air through turbulence mixing.

### RELATED APPLICATIONS

The present application is related to co-pending U.S. patent application Ser. No. 17/709,140 entitled: “WEARABLE AIR CLEANER WITH ULTRAVIOLET LIGHT DISINFECTION”, filed on Mar. 30, 2022, which application claims priority to U.S. patent application Ser. No. 17/159,099 entitled: “WEARABLE AIR CLEANER WITH ULTRAVIOLET LIGHT DISINFECTION”, filed on Jan. 26, 2021, now U.S. patent Ser. No. 11/318,221 granted on May 3, 2022, all these applications are incorporated herein by reference in their entireties.

### BACKGROUND

The COVID 19 Pandemic of 2020 has demonstrated to the World that we can be held hostage to an airborne pathogen. The Pandemic has also demonstrated just how quickly a new virus could sicken and kill large numbers of the human population in only a matter of months. Airborne viruses will continue to be a concern long after the COVID 19 Pandemic is over.

### SUMMARY

This invention is directed to a portable air sanitizing device that sterilizes airborne pathogens in the ambient air using UVC light and provides the sanitized air as a cone or cylinder of high velocity air to prevent mixing with untreated ambient air. In one embodiment, a nozzle directs the cone or cylinder of high velocity sanitized air at a user's mouth and nose so that the user can breathe in the sanitized air before it mixes with the untreated ambient air through turbulence mixing.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1 shows an aspect view of an example portable air sanitation device.

FIG. 2 shows an example portable air sanitation device as worn by a user.

FIG. 3A shows a cross-sectional view of an example portable air sanitation device.

FIG. 3B shows a front view of an example portable air sanitation device.

FIG. 3C shows a side cross-sectional view of an example portable air sanitation device with an air nozzle.

FIGS. 4-11 show examples of parabolic-shaped nozzles that may be used by a portable air sanitation device.

FIGS. 12-15 show examples of conical-shaped nozzles that may be used by a portable air sanitation device.

FIG. 16 shows a cross-sectional view of an example of an annular nozzle.

FIG. 17 shows a discharged airflow diagram of another example portable air sanitation device.

FIG. 18 shows a removable filter mount at the air inlet of an example portable air sanitation device.

FIG. 19 shows an exploded view of a removable filter mount of an example

portable air sanitation device.

FIG. 20 shows a cross-sectional view of an example portable air sanitation device.

FIG. 21 shows yet another example portable air sanitation device.

FIG. 22 shows an exploded view of portable air sanitation device.

FIG. 23A shows the top view of portable air sanitation device.

FIG. 23B is a cross-sectional view of portable air sanitation device as shown in FIG. 23A.

FIG. 24 shows a further example portable air sanitation device.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The Covid 19 Pandemic of 2020 has demonstrated to the world that we can be held hostage to an airborne pathogen. The Pandemic has also demonstrated to us just how quickly a new virus could sicken and kill large numbers of the human population in only a matter of months. Issues caused by airborne pathogens include lung disease, asthmatic disease, lung cancer, chronic obstructive bronchitis, myocardial infarction, vascular vasoconstriction, high blood pressure premature births, low birth weights, fetal anomalies, and infant mortality among others. Airborne pathogens are responsible for COVID 19, influenza, asthma, tuberculosis, whooping cough and the common cold, among others.

To combat future airborne pathogens, UVC energy can be used to render the new viruses sterile, as it does to all known airborne pathogens. The device described herein takes advantage of this phenomena, and could offer a solution to living in a contaminated world with full freedom of movement without needing to wear a mask.

The method of applying the UVC dose is by shining electromagnetic waves of the frequency of UVC on to the air stream. The level of UVC dose is a function of the optical output of the light source at the frequency of concern, as well as the distance between the light source and the virus, and the amount of time the virus is exposed to the UVC light. The equation for the dose is: D=(P*t)/(4πd²) where D is the dose in units such as milli-Joules/cm²; P is the UV Bulb Power in units such as milli-Watts; t is the exposure time in units such as seconds; and d is the distance in units such as cm.

This invention is directed to a portable air sanitizing device that sterilizes airborne pathogens in the ambient air using UVC light. The inventor designs the portable air sanitizing device so that the ambient air receives a sufficient dose of UVC light to sterilize airborne pathogens in that air. The inventor recognizes the need to prevent the treated air from the portable air sanitizing device from being contaminated with untreated ambient air before the treated air can be breathed in by a use. For this reason, the inventor designed the portable air sanitizing device to provide the sanitized air as a cone or cylinder of high velocity air to prevent mixing with untreated ambient air. In one embodiment, a nozzle directs the cone or cylinder of high velocity sanitized air at a user's mouth and nose so that the user can breathe in the sanitized air before it has the opportunity to mix with the untreated ambient air through turbulence mixing.

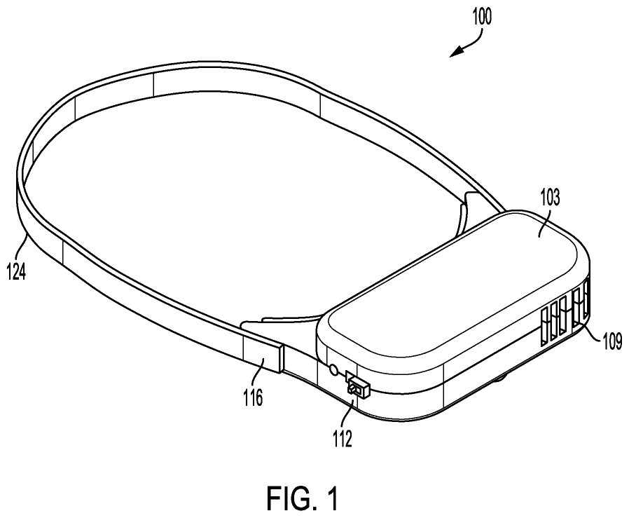

FIG. 1 shows an aspect view of an example portable air sanitation device 100. The portable air sanitation device 100 can be fastened to a wearable apparatus, such as a headband, a helmet, a visor, and a hat. In this embodiment, portable air sanitation device 100 is fastened to a headband 124 by attachment mechanism 116. Attachment mechanism 116 can be any removeable or permanent attachment mechanism, such as mechanical mechanism, magnetic, Velcro, adhesive and the like. Portable air sanitation device 100 includes housing 103, inlet 109, and control 112. Housing 103 serves as an enclosure to the components of portable air sanitation device 100. Inlet 109 allows ambient air to enter portable air sanitation device 100. Control 112 controls the electrical components in portable air sanitation device 100.

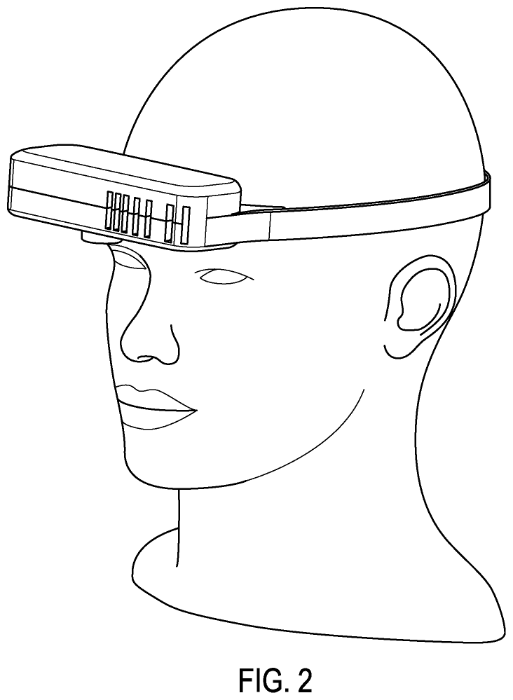

FIG. 2 shows the example portable air sanitation device 100 as worn by a user. Headband 124 enables portable air sanitation device 100 to be worn on the user's head.

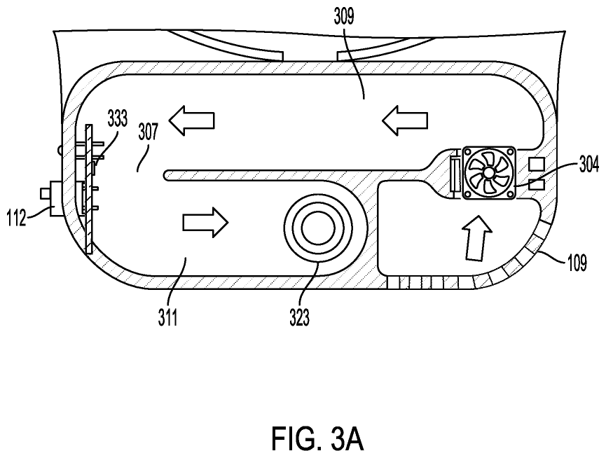

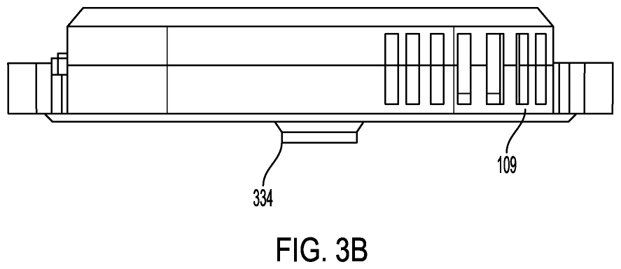

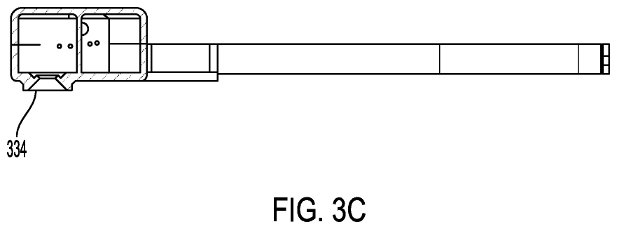

FIG. 3A shows a cross-sectional view of the example portable air sanitation device 100. The cross-sectional view allows components of portable air sanitation device 100 to be shown. These components include fan unit 304, chamber 307, air passage 309, air passage 311, UVC LED 333, and outlet 323.

Fan unit 304 pulls ambient air into portable air sanitation device 100 through inlet 109. The ambient air then passes through fan unit 304 along passage 309 into chamber 307. In chamber 307, UVC LED 333 provides a UVC light dose to the ambient air. The dose sterilizes the air from airborne pathogens. In one embodiment, chamber 307 is configured to slow the ambient air to a velocity such that the air will receive a sufficient dose to kill the airborne pathogens. The inventor found that a UVC ray dose of between 3 to 30 mJ/cm² is optimal for sterilizing the air. For that range of dosage, the inventor found that slowing the air velocity in chamber 307 to a range of between 0.1 to 40 inches per second provides good results. This slowing can be achieved by varying cross-sectional areas so that the cross-sectional area in chamber 307 is larger, which results in slower velocity for a given volumetric airflow. For example, the cross-sectional area of chamber 307 can be larger than the cross-sectional area of passage 309 and passage 311 to achieve the desired air velocity at each location.

The UVC light is harmful to skin and eyes so special care should be taken to prevent users from being exposed to the UVC light. Housing 103 and other components surrounding chamber 307 and UVC LED 333 should be made of materials that block UVC rays.

The sterilized air from chamber 307 of portable air sanitation device 100 should be discharged as a cone or cylinder of air with velocity high enough to prevent untreated ambient air from mixing with the sanitized air by minimizing turbulence prior to entering the mouth and nose of a user. To achieve this, the air velocity through passage 311 should be increased relative to the air velocity in chamber 307 by, for example, decreasing the cross-sectional area of passage 311 relative to the cross-sectional area of chamber 307. The inventor found that an air velocity of between 0.1 to 40 inches per second can be sufficient to impart enough air momentum and velocity at outlet 323 to achieve the desire discharge velocity at the air nozzle exit, depending on the UVC LED optical power.

Fan unit 304 and UVC LED 333 can be controlled by electronic circuitry and powered by a battery. Control 112 can be used to control fan unit 304 and UVC LED 333. Electronic circuitry can be incorporated into various parts of portable air sanitation device 100. In one embodiment, the battery is positioned at the back portion of headband 124 to achieve a balanced weight distribution. In another embodiment, the battery is worn by the user as a unit separate from headband 124 and connected to other components of portable air sanitation device 100 by a wire. The battery pack could be located on the user's belt or a nearby power source.

FIG. 3B shows a front view of the example portable air sanitation device 100. Untreated ambient air enters portable air sanitation device 100 through inlet 109. After the air is sanitized, the sanitized air is discharged through air nozzle 334. FIG. 3C shows a side cross-sectional view of the example portable air sanitation device 100 with air nozzle 334. In one embodiment, an aiming mechanism that directs the discharged cone or cylinder of high velocity air from the air nozzle to a user's mouth and nose. For example, air nozzle 334 can be attached to portable air sanitation device 100 with a rotatable coupling so that air nozzle 334 can be aimed. The discharged air from air nozzle exit 334 should have enough velocity to prevent untreated ambient air from mixing with the sanitized air by minimizing turbulence prior to entering the mouth and nose of a user. The range of airspeed is dependent on the specific nozzle design. The inventors found that a velocity range of between 20-200 inches per second yields good results.

An aspect of portable air sanitation device 100 is how the treated air is delivered to the user. The design of the air nozzle of portable air sanitation device 100 should discharge sanitized air into a tight cone or cylinder. This cone or cylinder of sanitized air can envelop the user's nose and mouth, preventing any untreated air from entering the user's mouth and nose. Such a high velocity cone or cylinder of treated air can eliminate the need for a face shield to achieve protection from untreated air. An optional face shield could provide more protection for the user.

Certain nozzle design enables the portable air sanitation device 100 to discharge the desired cone or cylinder of air with enough velocity to prevent untreated air from mixing with the air in the cone or cylinder and entering the user's mouth or nose. The inventor found that nozzle configurations of conical, parabolic, annular, and equivalent can create the desired zone of treated air for enveloping the users face and nose. Other nozzle designs may also be feasible.

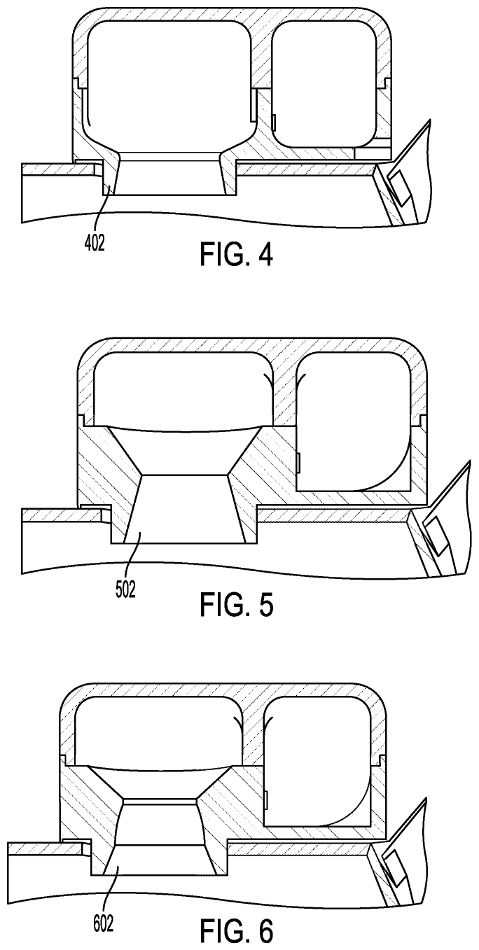

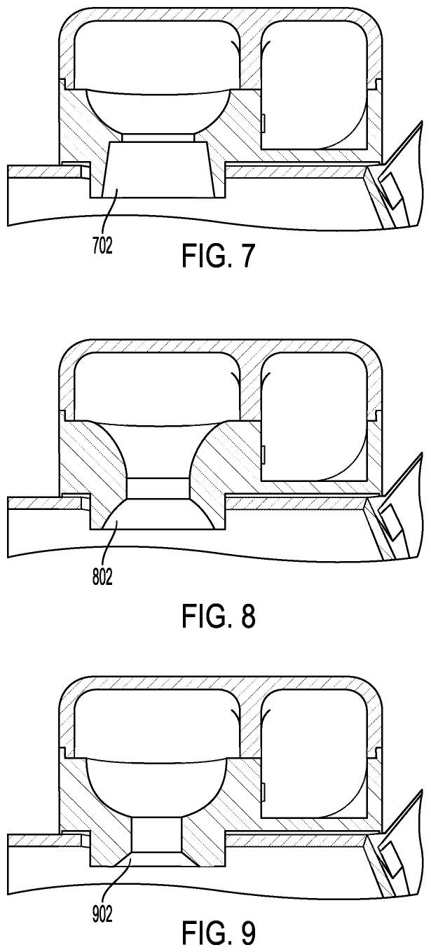

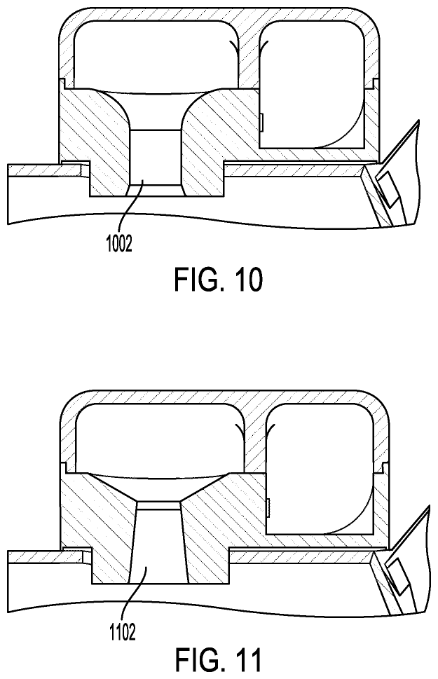

FIGS. 4-11 show examples of parabolic-shaped nozzles that may be used by portable air sanitation device 100. These parabolic-shaped nozzles include ASTAR nozzle 402, RAO nozzle 502, double bell nozzle 602, converging convex nozzle 702, converging concave nozzle 802, Moby Dick nozzle 902, Deich nozzle 1002, and LJ nozzle 1102.

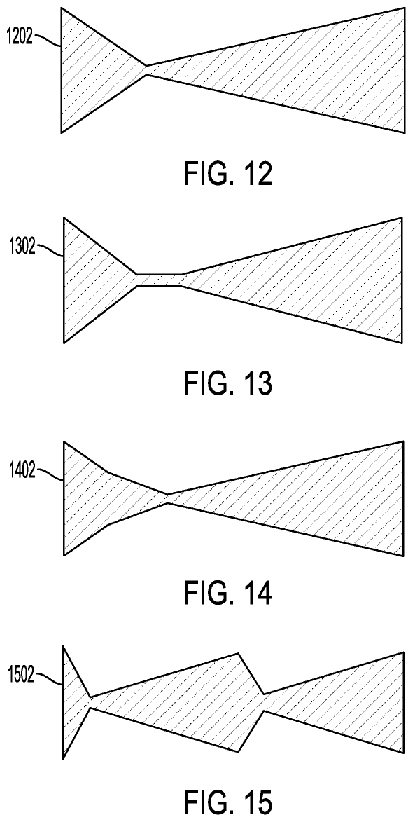

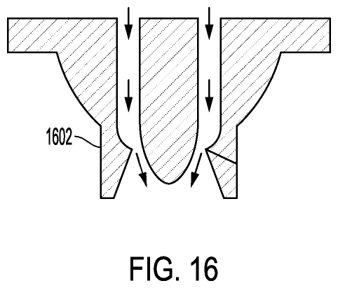

FIGS. 12-15 show examples of conical-shaped nozzles that may be used by portable air sanitation device 100. These conical-shaped nozzles include converging-diverging nozzle 1202, modified converging-diverging nozzle 1302, dual converging nozzle 1402, and multi-converging-diverging nozzle 1502. FIG. 16 shows a cross-sectional view of an example of an annular nozzle 1602.

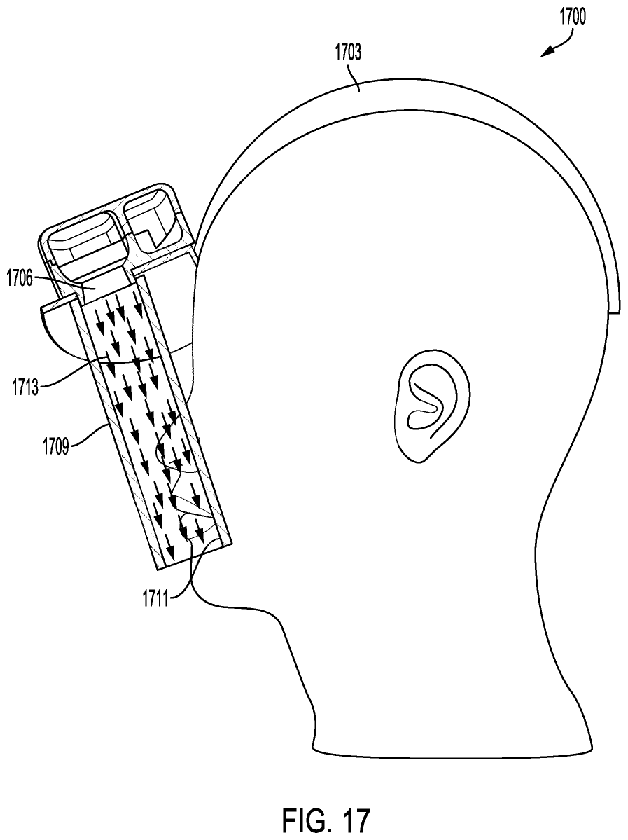

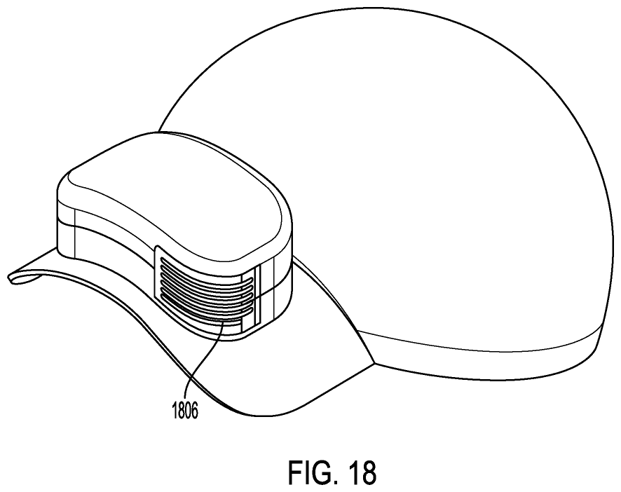

FIG. 17 shows a discharged airflow diagram of another example portable air sanitation device 1700. This example portable air sanitation device 1700 is attached to a hat 1703. Nozzle 1706 of portable air sanitation device 1700 discharges a cone of air illustrated by cone boundaries 1709, boundaries 1711 and air flow 1713.

Boundaries 1709 and boundaries 1711, illustrated as thicker lines, indicate the regions where the mixing of treated and untreated air will occur. This is called the shear layer, or the mixing layer. The air in this region will not be safe to breathe due to untreated air mixing with treated air. These regions should be kept away from the mouth and nose. The air will transfer from laminar flow to turbulent flow in this region. The more turbulent this area is, the more likely the air could do a 360 degree turn and head back into the higher velocity airstream with some horizontal momentum and potentially knock or bring an intreated air particle into the treated section. The thinner the shear layer is, the less likely the air will do a 360 degree turn and enter back into the treated air section as shown below. The configurations of portable air sanitation device 1700 described herein are designed to prevent such turbulent mixing before the cone of treated air reaches the user's mouth and nose.

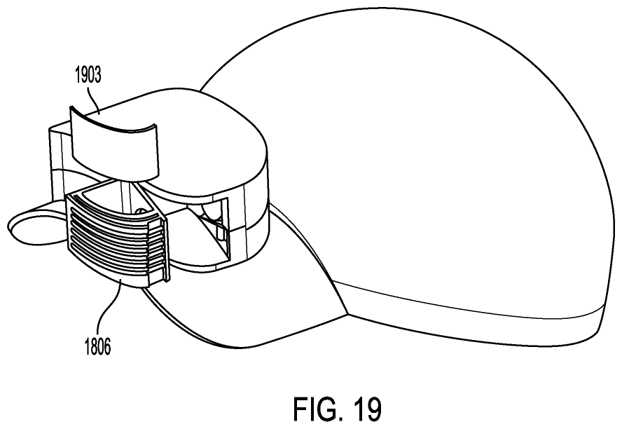

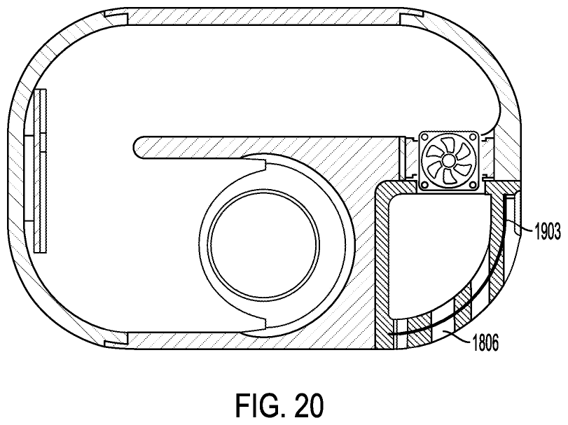

FIG. 18 shows a removable filter mount 1806 at the air inlet of the example portable air sanitation device 1700. FIG. 19 shows an exploded view of the removable filter mount 1806 of the example portable air sanitation device 1700. A replaceable filter 1903 can be placed into removable filter mount 1806. The material of replaceable filter 1903 can be of K95 or N95 material, or an alternative material which performs the function of filtering particles. FIG. 20 shows a cross-sectional view of the example portable air sanitation device 1700. The view shows example placement of filter mount 1806 and replaceable filter 1903.

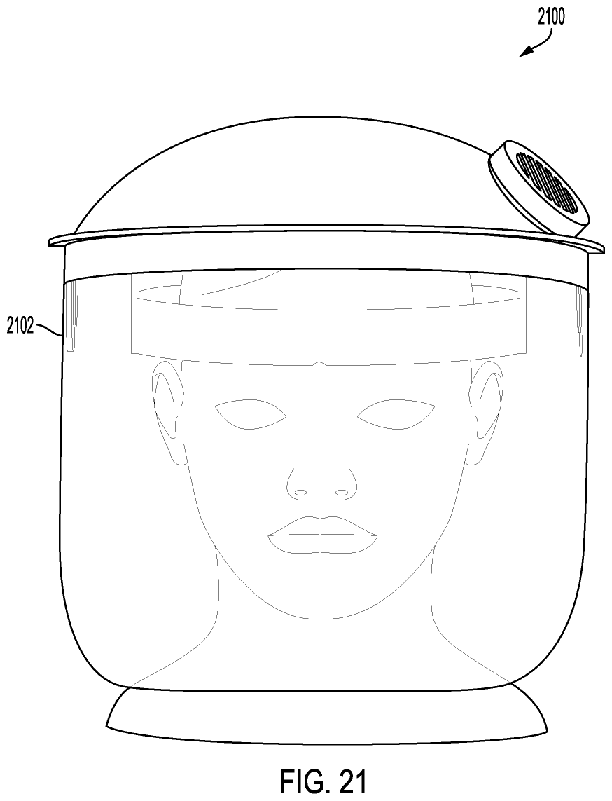

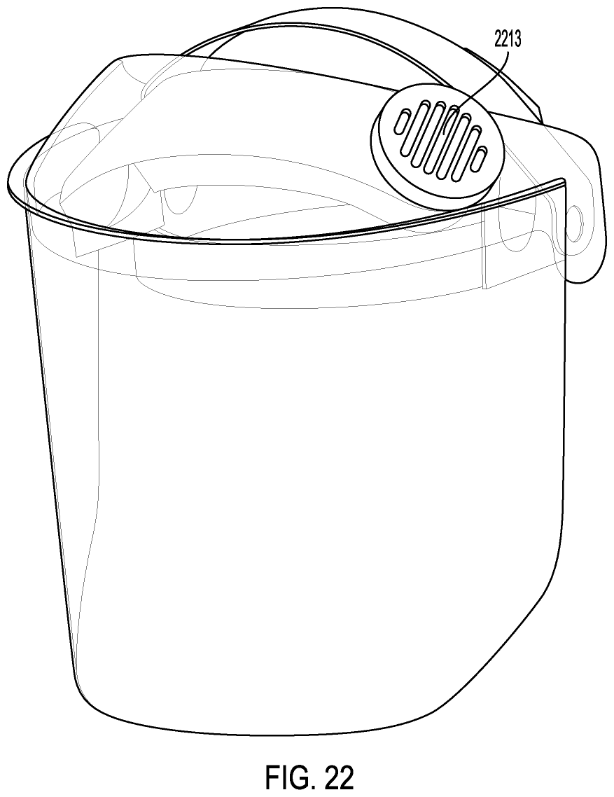

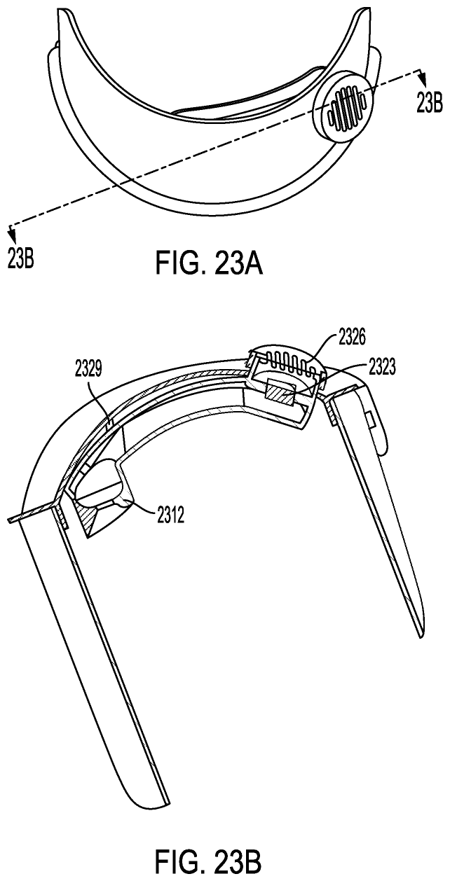

FIG. 21 shows yet another example portable air sanitation device 2100. This example portable air sanitation device 2100 is attached to face shield 2102. Face shield 2102 provides additional protection from the untreated ambient air resulting from air currents and wind. FIG. 22 shows an exploded view of portable air sanitation device 2100, which includes air inlet filter housing 2213. FIG. 23A shows the top view of portable air sanitation device 2100. FIG. 23B is a cross-sectional view of portable air sanitation device 2100 as shown in FIG. 23A. In this view, fan unit 2323, replaceable filter 2326, UVC LED 2329 and air nozzle 2312 are shown in their respective position in portable air sanitation device 2100.

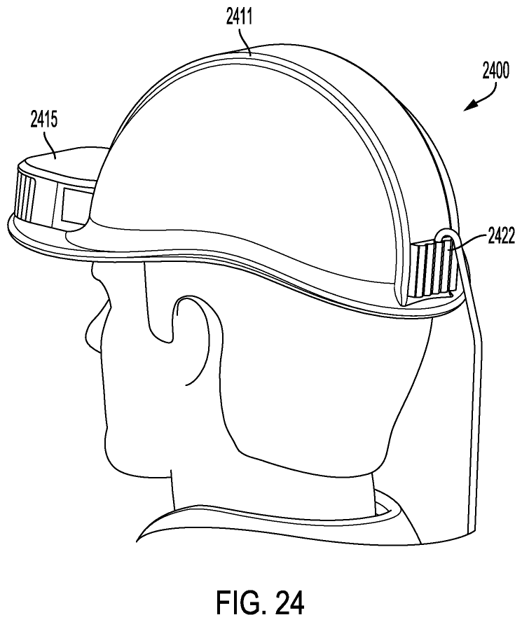

FIG. 24 shows a further example portable air sanitation device 2400. This example portable air sanitation device 2400 is attached to safety helmet 2411. Housing 2415 and associated components (such as inlet, UVC lights, fan, controller, etc.) of portable air sanitation device 2400 are positioned in the front of safety helmet 2411. In this embodiment, battery 2422 for powering example portable air sanitation device 2400 is mounted in the rear of safety helmet 2411 to balance the weight of other components. Battery 2422 can be a standalone unit or be serviced by an umbilical cord to provide longer duration, as well as charging battery 2422. The umbilical cord can be connected to a power supply, which can be a PC, a portable power supply mounted to the user's body, or from a source close to the user. Examples of these alternative power sources could be a PC on the floor or table, a mobile power supply mounted on a belt, a vest or the like. The figure below shows the user of the device with a waist mounted mobile power supply.

In conclusion, a portable air sanitizing device that sterilizes airborne pathogen in the ambient air using UVC light is described herein. The portable air sanitizing device is designed so that the ambient air receives a sufficient dose of UVC light to sterilize airborne pathogens in that air. The portable air sanitizing device prevents the treated air from the portable air sanitizing device from being contaminated with untreated ambient air before the treated air can be breathed in by a use.

The inventor believes that the ability to walk freely with additional protection from particulates and pathogens will have immediate impact for those seeking safer air to breathe. The portable air sanitizing device described herein provides a way for the immunocompromised, people who suffer from certain allergies, people who want additional protection from airborne pathogens and particulates, people who want immediate relief from wildfire smoke or air pollution, and people who want additional protection from the cold or flu virus, to experience mobility with more peace of mind. It is an alternative to a mask that could be shown to be more effective against particulates and pathogens.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claims require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A portable air sanitizing device comprising:
an inlet that accepts ambient air into the portable air sanitizing device;
a fan unit that pulls the accepted ambient air from the inlet;
a chamber with an LED light, the chamber accepting the ambient air pulled in by the fan unit and sanitizing the ambient air by illuminating the ambient air with UVC rays from the LED light; and
an air nozzle that discharges the sanitized air from the chamber as a cone or cylinder of high velocity air.

2. The portable air sanitizing device of claim 1, wherein the air nozzle is configured as at least one type of a group comprising parabolic-shaped nozzle, conical-shaped nozzle, annular nozzle, ASTAR nozzle, RAO nozzle, double bell nozzle, converging convex nozzle, Moby Dick nozzle, Deich nozzle, LJ nozzle, converging-diverging nozzle, modified converging-diverging nozzle, dual converging nozzle, and multi-converging-diverging nozzle.

3. The portable air sanitizing device of claim 1, wherein the air nozzle is configured to discharge the sanitized air with a velocity in a range of between 20 inches per second to 200 inches per second.

4. The portable air sanitizing device of claim 1, further comprising an aiming mechanism that directs the discharged cone or cylinder of high velocity air from the air nozzle to a user's mouth and nose.

5. The portable air sanitizing device of claim 1, wherein the chamber is configured to slow the ambient air pulled in by the fan unit to a velocity in a range of between 0.1 inches per second to 40 inches per second.

6. The portable air sanitizing device of claim 1, further comprising an attachment mechanism that fastens the portable air sanitizing device to a wearable apparatus.

7. The portable air sanitizing device of claim 6, wherein the attachment mechanism is configured to removably fasten the portable air sanitizing device to a wearable apparatus.

8. The portable air sanitizing device of claim 6, wherein the wearable apparatus includes at least one type of a comprising a headband, a helmet, a visor, a face shield, and a hat.

9. The portable air sanitizing device of claim 1, wherein the inlet further comprising a filter mount that accepts a removable air filter.

10. The portable air sanitizing device of claim 1, further comprising a face shield.

11. A portable air sanitizing device comprising:
means for pulling ambient air into a chamber in the portable air sanitizing device;
means for sanitizing the ambient air using UVC rays in the chamber; and
means for discharging the sanitized air from the chamber as a cone or cylinder of air with velocity high enough to prevent untreated ambient air from mixing with the sanitized air by minimizing turbulence prior to entering mouth and nose of a user.

12. The portable air sanitizing device of claim 11, wherein the means for discharging the sanitized air further includes means for directing a mixing region of the discharged air and untreated ambient air away from the mouth and nose of the user.

13. The portable air sanitizing device of claim 11, wherein the means for sanitizing the ambient air using UVC rays further comprising means for slowing the pulled air in the chamber to a velocity sufficient for providing a UVC ray dose of 3-30 mJ/cm2.

14. The portable air sanitizing device of claim 11, further comprising a face shield and means for attaching the field shield to protect the user from the untreated ambient air resulting from air currents and wind.

15. The portable air sanitizing device of claim 11, further comprising means for filtering the pulled ambient air with a removable air filter.

16. A method for providing sanitized air to a user of a portable device comprising:
pulling ambient air into a chamber of the portable device;
sanitizing the pull ambient air using UVC rays in the chamber; and
discharging the sanitized air from the chamber as a cone or cylinder of air with velocity high enough to prevent untreated ambient air from mixing with the sanitized air by minimizing turbulence prior to entering mouth and nose of a user.

17. The method of claim 16, further comprising directing a mixing region of the discharged air and untreated ambient air away from the mouth and nose of the user.

18. The method of claim 16, further comprising slowing the pulled air in the chamber to a velocity sufficient for providing a UVC rays dose of 3-30 mJ/cm2.

19. The method of claim 16, further comprising attaching a face shield to protect the user from the untreated ambient air resulting from air currents and wind.

20. The method of claim 16, further comprising filtering the pulled ambient air with a removable air filter.
